(19) United States
(12) Patent Application Publication
(10) Pub. No.: US 2008/0234787 A1
(43) Pub. Date: Sep. 25, 2008

(54) Laser treatment procedure atlas

(76) Inventors: Anat Kaphan, Halonim (IL); Lora Varnelis, Vacaville, CA (US); David Weinstein, Youvalim (IL)

Correspondence Address: MINTZ, LEVIN, COHN, FERRIS, GLOVSKY AND POPEO, P.C;ATTN: PATENT INTAKE CUSTOMER NO. 30623, ONE FINANCIAL CENTER, BOSTON, MA, 02111, US

(21) Appl. No.: 11/974,404

(22) Filed: Oct. 11, 2007

Related U.S. Application Data
(60) Provisional application No. 60/850,636, filed on Oct. 11, 2006.

(51) Int. Cl. A61N 5/067

(52) U.S. Cl. 607/89

(57) ABSTRACT

A user interface for a general purpose laser treatment system is provided. Generally, the user interface may be associated with a carbon dioxide (CO2) laser for use in various surgical procedures, for example, veterinary or medical surgical procedures. A treatment atlas including a collection of treatment procedures is stored within a database structure. The treatment procedures includes laser settings associated with each of the treatment procedures. The interface can provide a recommended treatment procedures to a user based on required application. The treatment atlas can be accessed through the internet and requested treatment procedures (and the corresponding laser settings) can be downloaded to a laser treatment system. The treatment atlas can also be a component of a Web 2.0 social networking system to enable users and system administrators to update and modify the collection of treatment procedures.

### CROSS-REFERENCE TO RELATED ACTIONS

This application claims the priority of U.S. Provisional Application No. 60/850,636 filed on Oct. 11, 2006.

### BACKGROUND

General purpose medical and veterinary laser systems may be accompanied by a user interface. Current user interfaces may enable a user to change laser settings, for example, pulse type, frequency, number of cycles, pulse duration, resting time between pulses, etc. The user interface may have a memory for storing the information and/or settings relating to a practitioner's previously performed procedures, e.g., for a particular patient, or for a procedure that the practitioner has performed. These stored settings may be searchable by treatment name or patient name. These interfaces are limited, and there is a need for an improved user interface.

### SUMMARY

In general, in an aspect, the invention provides an apparatus for selecting laser settings in a laser treatment system including a storage device, an input and output component configured for presenting and receiving information to and from a user, and a processor programmed to maintain in the storage device a database identifying treatment procedures and the corresponding laser settings for each of the treatment procedures, display a plurality of the treatment procedures on the input and output component, receive a selected treatment procedure from the user, and set the laser settings in the laser treatment system based on the selected treatment procedure.

Implementations of the invention may include one or more of the following features. A communication port operably connected to the Internet. The processor can be configured to receive the database identifying treatment procedures and the corresponding laser settings for each of the treatment procedures via the communication port. The laser settings can include at least one of the group consisting of power level, pulse type, pulse frequency, pulse duration, and resting time between pulses. The database can further identify a textual description of the treatment procedure, and the processor can be programmed to display the textual description associated with the selected treatment procedure. The textual description can include at least one of the group consisting of a general description of the treatment procedure, techniques, preoperative considerations, anesthesia types which may be used, procedure preparations, equipment, proposed laser settings which may be used with the specific procedure, closure techniques, postoperative care, follow up procedures, and general comments about the treatment procedure. The database can further identify a pictorial description of the treatment procedure, and the processor can be is programmed to display the pictorial description associated with the selected treatment procedure. The processor can be programmed to highlight at least one of the displayed plurality of treatment procedures based on a recommendation algorithm.

In general, in another aspect, the invention provides a computer-readable medium having computer-executable instructions for performing a method including maintaining a database identifying a plurality of treatment procedures and the corresponding laser settings for each of the treatment procedures, displaying at least one treatment procedure to a user, receiving a selected treatment procedure from the user, and controlling a laser treatment device based on the laser settings corresponding to the selected treatment procedure.

Implementations of the invention may include one or more of the following features. Computer-executable instruction for receiving an application requirement from the user, executing a recommendation algorithm based on the application requirement, and displaying at least one treatment procedure to the user based on results of the recommendation algorithm. Computer-executable instructions for receiving at least one laser setting from the user. The database further can identify a textual description for each of the treatment procedures. The database can further identify at least one pictorial description for at least one of the treatment procedures.

In general, in another aspect, the invention provides an article of manufacture including a computer-readable medium having stored thereon a data structure, a first field containing data representing a treatment procedure associated with a laser treatment device, and a second field containing data representing at least one laser setting, wherein the laser setting is a parameter associated with the laser treatment device.

Implementations of the invention may include one or more of the following features. A third field containing data representing a textual description of the treatment procedure. A pictorial field containing data representing a pictorial description of the treatment procedure. A username field containing data representing a user of the laser treatment device.

In general, in another aspect, the invention provides in a computer system having a graphical using interface including a display and selection indicator, a method of providing and selecting from a menu on the display, the method including retrieving a set of menu entries for the menu, each of the menu entries representing a treatment procedure associated with a laser treatment device, wherein the treatment procedure corresponds to a plurality of laser settings, and the laser treatment device is configured to receive the laser settings, displaying the set of menu entries on the display, receiving a menu entry selection signal indicative of the position of the selection indicator within the set of menu entries, and in response to the signal, sending the laser settings corresponding to the menu entry selection to the laser treatment device.

Implementations of the invention may include one or more of the following features. Receiving a view selection signal indicative of a user pressing a view toggle button, and in response to the view selection signal, displaying a textual description of the treatment procedure associated with the menu entry selection signal. Receiving a view selection signal indicative of a user pressing a view toggle button, and in response to the view selection signal, displaying a pictorial description of the treatment procedure associated with the menu entry selection signal.

These and other capabilities of the invention, along with the invention itself, will be more fully understood after a review of the following figures, detailed description, and claims.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is an illustration of a user interface treatment screen according to one embodiment of the present invention;

FIG. 2 is an illustration of a procedure atlas screen according to one embodiment of the present invention;

FIG. 3 is an illustration of a procedure description screen according to one embodiment of the present invention; and

FIG. 4 is an illustration of a procedure description screen in the picture description display condition, according to one embodiment of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF PREFERRED EMBODIMENTS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The present invention provides a user interface for a general purpose laser treatment system. Typically, the user interface according to the present invention may be associated with a carbon dioxide (CO2) laser for use in various surgical procedures, for example, veterinary or medical surgical procedures.

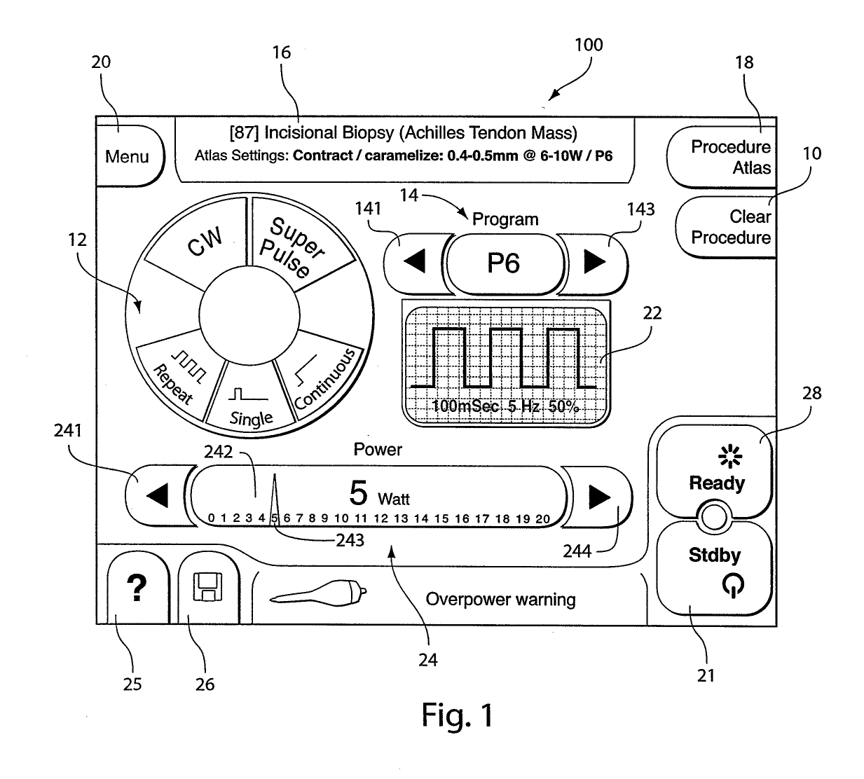

Reference is now made to FIG. 1, which is an illustration of a user interface treatment screen (e.g., application program interface) 100 according to one embodiment of the present invention. Treatment screen 100 may be a touch-screen for selecting the laser setting for a specific treatment. It will be understood that embodiments of the invention include any input/output device for presenting information to a user and receiving information therefrom. Treatment screen 100 may enable selecting a desired pulse type of the laser for a specific treatment procedure, for example, by pulse selection buttons 12. Pulse selection buttons 12 may include, for example, assembly of touch. buttons, each button for a certain pulse type, e.g., continuous wave (CW), super pulse, repeat pulse, single pulse, continuous pulse, and/or any combination thereof. Touching a touch button may set the system to operate the laser by the selected pulse type. One or more touch buttons may be selected simultaneously. Combination of certain selected buttons may set the pulse to a required pulse type.

Treatment screen 100 may enable selecting of the desired laser power level for a specific treatment procedure, for example, by power selection buttons 24. Power selection buttons 24 may include a power ruler 242. Selection of the power level may be done by touching power ruler 242, thus, for example, placing a cursor 243 on a desired power level. Power selection buttons 24 may further include, for example, arrow buttons 241 and 244, for decreasing or increasing the power level, respectively.

Treatment screen 100 may include pulse display 22. Pulse display 22 may display the pulse shape together with other pulse data (e.g., parameters) such as, for example, frequency, number of cycles, pulse duration, resting time between pulses, etc. Certain combinations of pulse characteristics may be stored as pulse programs, which may be selected by the user, for example, by program selection buttons 14. Program selection buttons 14 may include, for example, arrow buttons 141 and 143 for selecting a desired program.

The user may store a combination of laser settings for future use, for example, by touching the save button 26. When the user has completed to set the desired laser setting and is ready to start the operation, he may touch the ready button 28, to lock the chosen settings and start using the laser. In some embodiments of the invention in which a user is connected to the Internet, the save button or a different button may transmit the user's preferences for a particular operation to a central server suitable for collecting information from various users for settings used for a variety of operations.

Treatment screen 100 may also include a clear button 10 to clear the procedure settings, a standby button 21 to set the user interface on a standby status, a menu button 20 to reach the menu of the user interface, and a help button 25 to receive help in operation of the user interface.

Embodiments of the laser treatment atlas may include a processor and memory configured to execute a programmed modifiable surgical procedure atlas, which may be accessed by the user interface, for example, through treatment screen 100, for example, by touching a procedure atlas button 18. In another example, the atlas can be installed on a personal computer which is operable coupled to a laser treatment device. As described herein below, the procedure atlas may include collection of proposed treatment procedures, based on, for example, research and/or case studies done by professionals. The atlas may be provided pre-installed on to a storage device by the manufacturer of the laser system, or may be a software program or database to be installed modularly thereon by a vendor. In general, the software program and atlas can be provided in a computer-readable medium such a computer memory, floppy disks, conventional hard disks, CD-ROMS, Flash ROMS, nonvolatile ROM, and RAM. In an example, the atlas system may be available on a local server and accessible to the laser system. In some embodiments of the invention, the atlas may be available on the Internet and accessible to the laser system, for example, on a subscription basis or by entering a username and password. Further, the atlas includes a recommendation algorithm configured to display or download a list of treatment procedures based on the users application requirements.

In an embodiment, the surgical procedure atlas is a database component on a Web 2.0 Social Networking system (e.g., Kwiqq, Onesite). The social networking system enables users to search and access the collection of treatment procedures, as well as exchange professional information about the procedures within the atlas. For example, a user may create or modify a treatment procedure and then save that procedure in the procedure atlas. Additionally, users can add other content such as textual descriptions and multimedia files (e.g., photos, video, voice recordings) to the atlas. The social networking system is configured to receive rating information and other meta-data about the specific procedures within the atlas, as well as enable searching and sorting on the meta-data.

Upon entering the procedure atlas, the user may be presented with a list of available procedures. As described below, the proposed treatment procedures may include proposed laser setting for a specific procedure, and the user may choose to automatically set the laser according to the proposed settings. The chosen procedure and the proposed laser settings for the chosen procedure may be displayed on treatment screen 100, for example, on procedure display 16. It will be noted that the user may alter the suggested laser settings and choose alternative ones according to his preferences and/or experience.

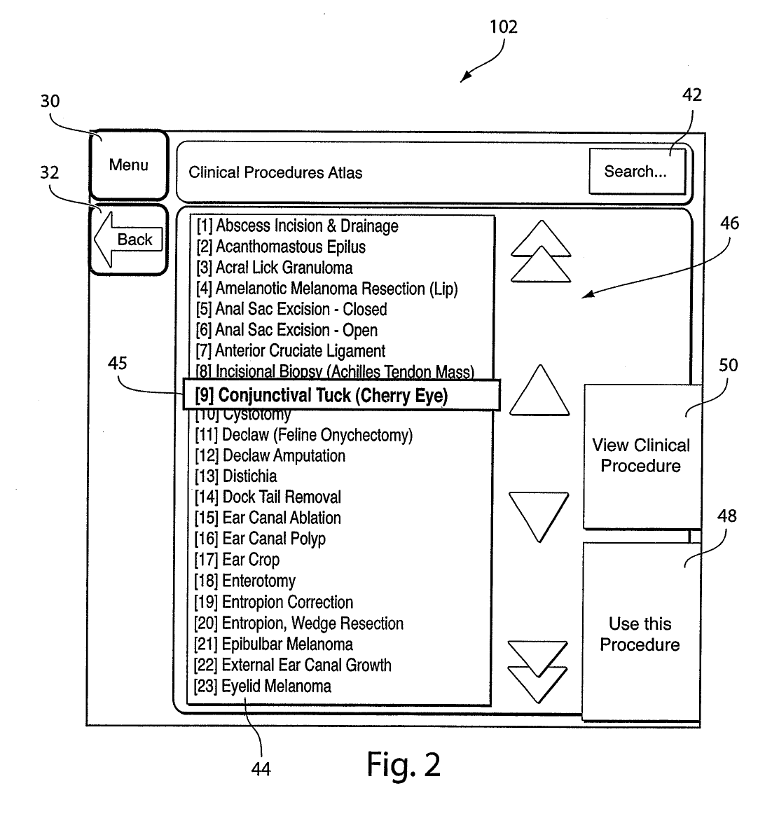

Reference is now made to FIG. 2, which is an illustration of a procedure atlas screen 102 according to one embodiment of the present invention, which may be reached by touching procedure atlas button 18 on treatment screen 100 as described herein above, or through the menu of the user interface. Procedure atlas screen 102 may include a list of surgical procedures 44. Indicator 45 may indicate the current chosen procedure. The user may choose a desired procedure by shifting indicator 45 to a specific procedure, for example, by arrow buttons 46. The user may also search for a desired procedure by touching the search button 42 and entering the desired procedure name, category or catalog number of the procedure.

Upon locating the appropriate procedure, the user may choose to either use or view the procedure listed.

If already familiar with the procedure, the user may simply select to use the settings stored for the procedure by touching the use button 48. The user may also view further description of the chosen procedure and the proposed settings by touching the view button 50.

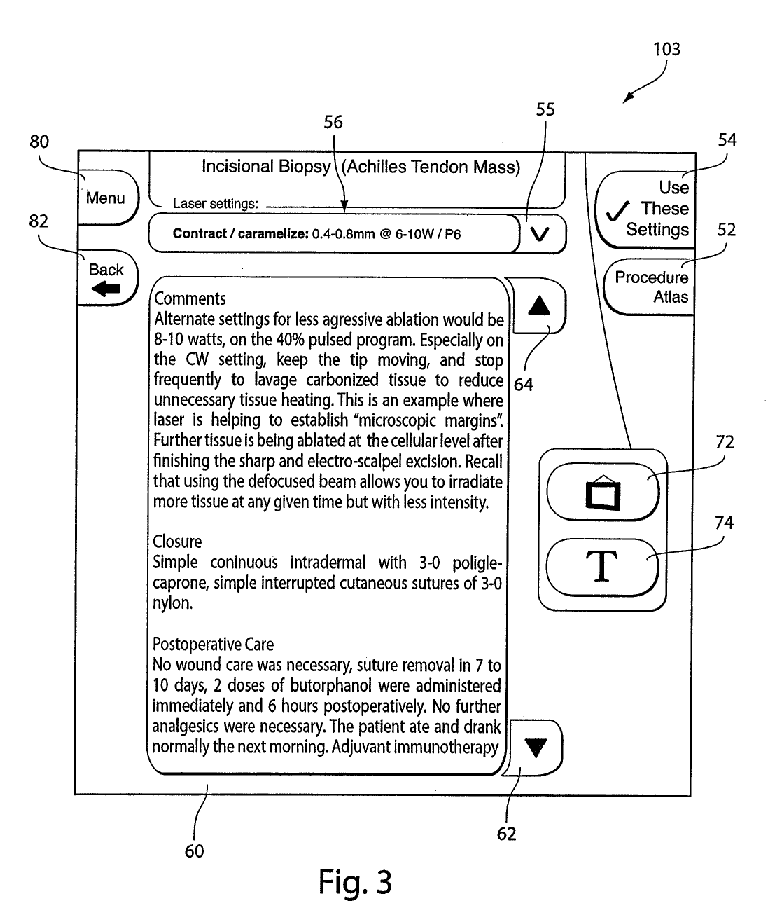

Reference is now made to FIG. 3, which is an illustration of a procedure description screen 103 according to one embodiment of the present invention. The screen of FIG. 3 may be reached, for example, by touching view button 50 on procedure atlas screen 102 as described herein above. On procedure description screen 103, the user may toggle between a textual description of the procedure (button 74) or a pictorial description (button 72).

FIG. 3 shows, for illustration, the text description display. Text description of the chosen procedure may be displayed on text display 60. The text may be scrolled up and down by text scrolling buttons 64 and 62, respectively. The text description of the proposed treatment procedure may include, for example, general description of the procedure, techniques, preoperative considerations, anesthesia types which may be used, procedure preparations, equipment, proposed laser settings which may be used with the specific procedure, closure techniques, postoperative care, follow up procedures, and other comments and notes with respect to the procedure. In some embodiments of the invention, the descriptions of the proposed treatment procedures may further include description of the patient and the histopathology of the treated tissue in the specific case study. The laser settings proposed by the atlas may include the proposed laser diameter or proposed range of laser diameters, the proposed power level range, and the pulse type that may be used. There may be different optional proposed laser settings, for example, for different types of patients or different steps of the operation.

Procedure description screen 103 may show the proposed laser settings for the chosen procedure on laser settings display 56. The user may choose between different optional proposed settings, for example, for different types of patients or different steps of the operation, for example, by arrow button 55. The user may set the laser settings by touching the use these settings button 54. The user may return to procedure atlas screen 102 by touching procedure atlas button 52. The user may return to the previously viewed screen by pushing the back button 82. Procedure description screen 103 may also include a menu button 80 to reach the menu of the user interface.

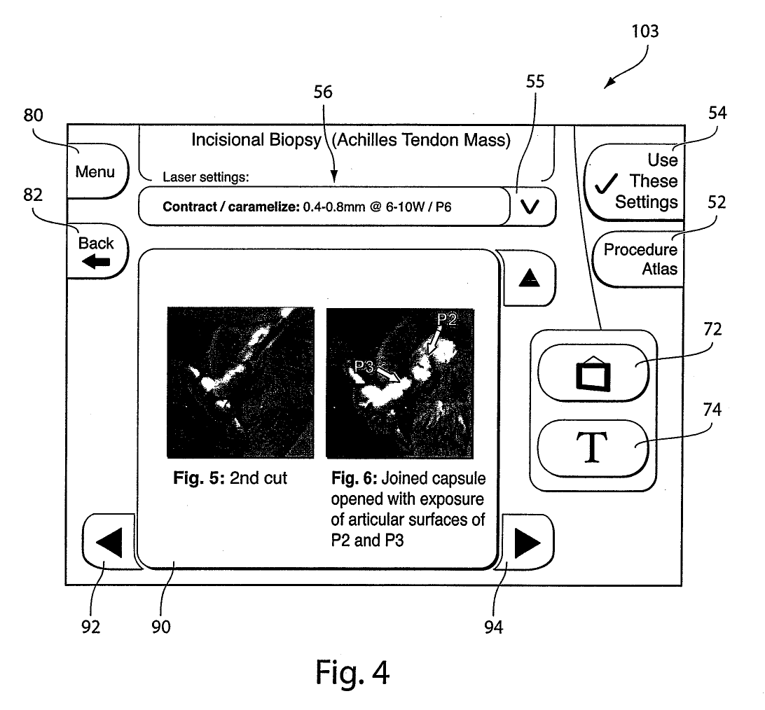

Reference is now made to FIG. 4, which is an illustration of procedure description screen 103 in the picture description display condition, according to one embodiment of the present invention. Picture description of the chosen procedure may be displayed on picture display 90. Picture display 90 may show pictures illustrating steps in the procedure in a case study. Picture display 90 may include short text descriptions of the displayed pictures. The displayed pictures may be scrolled by, for example, pictures scrolling buttons 92 and 94.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

Upon selection of a procedure, the user may be taken back to the main menu of FIG. 1 and the suggested settings may be selected by default. It will be noted that the procedure may be a multi-stage procedure, requiring different settings for the different stages of the operation. The user may be prompted to indicate when each stage is complete, at which time the system proceeds to set the laser settings as suitable for the next stage. In some embodiments, the user may modify the configuration of various procedures to reflect personal preferences for those procedures.

It will be recognized that the above is a description of merely some embodiments of the invention, but that the invention is not limited in this regard, and that other variations are possible within the spirit of the present invention.

Other embodiments are within the scope and spirit of the invention. For example, due to the nature of software, functions described above can be implemented using software, hardware, firmware, hardwiring, or combinations of any of these. Features implementing functions may also be physically located at various positions, including being distributed such that portions of functions are implemented at different physical locations.

Further, while the description above refers to the invention, the description may include more than one invention.

What is claimed is:

1. An apparatus for selecting laser settings in a laser treatment system, comprising:
a storage device;
an input and output component configured for presenting and receiving information to and from a user; and
a processor programmed to:
maintain in the storage device a database identifying treatment procedures and the corresponding laser settings for each of the treatment procedures,
display a plurality of the treatment procedures on the input and output component,
receive a selected treatment procedure from the user, and
set the laser settings in the laser treatment system based on the selected treatment procedure.

2. The apparatus of claim 1 further comprising a communication port operably connected to the Internet.

3. The apparatus of claim 2 wherein the processor is configured to receive the database identifying treatment procedures and the corresponding laser settings for each of the treatment procedures via the communication port.

4. The apparatus of claim 1 wherein the laser settings include at least one of the group consisting of power level, pulse type, pulse frequency, pulse duration, and resting time between pulses.

5. The apparatus of claim 1 wherein the database further identifies a textual description of the treatment procedure, and the processor is programmed to display the textual description associated with the selected treatment procedure.

6. The apparatus of claim 5 wherein the textual description includes at least one of the group consisting of a general description of the treatment procedure, techniques, preoperative considerations, anesthesia types which may be used, procedure preparations, equipment, proposed laser settings which may be used with the specific procedure, closure techniques, postoperative care, follow up procedures, and general comments about the treatment procedure.

7. The apparatus of claim 1 wherein the database further identifies a pictorial description of the treatment procedure, and the processor is programmed to display the pictorial description associated with the selected treatment procedure.

8. The apparatus of claim 1 wherein the processor is programmed to highlight at least one of the displayed plurality of treatment procedures based on a recommendation algorithm.

9. A computer-readable medium having computer-executable instructions for performing a method comprising:
maintaining a database identifying a plurality of treatment procedures and the corresponding laser settings for each of the treatment procedures;
displaying at least one treatment procedure to a user;
receiving a selected treatment procedure from the user; and
controlling a laser treatment device based on the laser settings corresponding to the selected treatment procedure.

10. The computer-readable medium of claim 9 further comprising computer-executable instruction for receiving an application requirement from the user, executing a recommendation algorithm based on the application requirement, and displaying at least one treatment procedure to the user based on results of the recommendation algorithm.

11. The computer-readable medium of claim 9 further comprising computer-executable instructions for receiving at least one laser setting from the user.

12. The computer-readable medium of claim 9 wherein the database further identifies a textual description for each of the treatment procedures.

13. The computer-readable medium of claim 9 wherein the database further identifies at least one pictorial description for at least one of the treatment procedures.

14. An article of manufacture comprising:
a computer-readable medium having stored thereon a data structure;
a first field containing data representing a treatment procedure associated with a laser treatment device; and
a second field containing data representing at least one laser setting, wherein the laser setting is a parameter associated with the laser treatment device.

15. The article of manufacture of claim 14 further comprising a third field containing data representing a textual description of the treatment procedure.

16. The article of manufacture of claim 14 further comprising a pictorial field containing data representing a pictorial description of the treatment procedure.

17. The article of manufacture of claim 14 further comprising a username field containing data representing a user of the laser treatment device.

18. In a computer system having a graphical using interface including a display and selection indicator, a method of providing and selecting from a menu on the display, the method comprising;
retrieving a set of menu entries for the menu, each of the menu entries representing a treatment procedure associated with a laser treatment device, wherein the treatment procedure corresponds to a plurality of laser settings, and the laser treatment device is configured to receive the laser settings;
displaying the set of menu entries on the display;
receiving a menu entry selection signal indicative of the position of the selection indicator within the set of menu entries; and
in response to the signal, sending the laser settings corresponding to the menu entry selection to the laser treatment device.

19. The method of claim 18 further comprising receiving a view selection signal indicative of a user pressing a view toggle button, and in response to the view selection signal, displaying a textual description of the treatment procedure associated with the menu entry selection signal.

20. The method of claim 18 further comprising receiving a view selection signal indicative of a user pressing a view toggle button, and in response to the view selection signal, displaying a pictorial description of the treatment procedure associated with the menu entry selection signal.
